(19) United States
(12) Patent Application Publication
(10) Pub. No.: US 2015/0190456 A1
(43) Pub. Date: Jul. 9, 2015

(54) USE OF CASPASE 3 ACTIVITY AND ITS INHIBITORS

(71) Applicants: Chuan-Yuan Li, Chapel Hill, NC (US); Qian Huang, Shanghai (CN); Fang Li, Durham, NC (US)

(72) Inventors: Chuan-Yuan Li, Chapel Hill, NC (US); Qian Huang, Shanghai (CN); Fang Li, Durham, NC (US)

(73) Assignee: THE REGENTS OF THE UNIVERSITY OF COLORADO, Denver, CO (US)

(21) Appl. No.: 14/128,228

(22) PCT Filed: Jun. 20, 2012
(86) PCT No.: PCT/US12/43227

§ 371 Date: Jan. 15, 2015

Related U.S. Application Data
(60) Provisional application No. 61/499,094, filed on Jun. 20, 2011.

(51) Int. Cl. A61K 38/00; G01N 33/574; A61K 31/5377; A61K 31/27; A61K 45/06; A61K 31/404

(52) U.S. Cl. CPC A61K 38/005; A61K 45/06; A61K 31/404; A61K 31/5377; A61K 31/27; G01N 33/5743; G01N 2333/96466

(57) ABSTRACT

The present invention provides methods for using the level of caspase 3 activity for determining or prognosticating likelihood of cancer recurrence or relapse in a patient after the treatment for cancer. The present invention also provides methods for treating cancer in a patient using a caspase 3 inhibitor in conjunction with a conventional cancer treatment.

### CROSS-REFERENCE TO RELATED APPLICATIONS

This application claims the priority benefit of U.S. Provisional Application No. 61/499,094, filed Jun. 20, 2011, which is incorporated herein by reference in its entirety.

### STATEMENT REGARDING FEDERALLY FUNDED RESEARCH

This invention was made with government support under grant numbers CA136748 and CA131408 awarded by the National Institutes of Health. The government has certain rights in the invention.

### FIELD OF THE INVENTION

The present invention relates to a method of determining a likelihood of cancer recurrence in a patient. In particular, the invention relates to determining the level of active caspase 3 from a cancer cell sample to determine the likelihood of recurrence of cancer in the patient as well as determining the cancer treatment protocol.

### BACKGROUND OF THE INVENTION

Early detection and treatment of cancers leads to better patient survival. For example, if breast cancer is detected early, e.g., prior to spreading to other areas, the five-year survival rate can be greater than 90%. For lung cancer, when it is detected as a single mass the 5-year survival is more than 45%. When it has spread, the five-year survival is less than 15%. For cervical cancer, additional improvement in survival occurs when pre-cancerous changes are found and treated before developing into a more severe stage.

While these data show benefits of early detection and treatment, there are patients that experience cancer recurrence or relapse even after early detection and treatment. Unfortunately, to date there is no test that can determine which patient will experience relapse or recurrence of cancer after treatment. Thus, all cancer patients are routinely monitored after a successful treatment. It would be helpful for doctors and healthcare providers to have a reliable and accurate test that can identify which cancer patient will likely experience relapse or recurrence of cancer in order to more closely monitor those patients.

Accordingly, there is a need for a method that can reliably and accurately predict which cancer patient will likely experience relapse or recurrence of cancer.

### SUMMARY OF THE INVENTION

Some aspects of the invention provide methods for determining likelihood of cancer recurrence or relapse in a cancer patient undergoing or have completed a cancer treatment. In particular, one embodiment of the invention provides a method for predicting cancer recurrence in a subject comprising determining the amount of cells with caspase 3 activity from a cell sample obtained from the subject's cancerous tissue or organ. If the amount of cells having active caspase 3 is at least 5%, typically at least 10%, and often at least 15%, then the patient is likely to experience recurrence or relapse after a cancer treatment therapy. In some embodiments, the cell sample comprises a tumor biopsy sample of the subject or a blood or serum sample of the subject. Yet in other embodiments, cancer comprises squamous cell carcinoma. Still in other embodiments, cancer comprises breast cancer, prostate cancer, colon cancer, melanoma, liver cancer, leukemia, lymphoma, and other solid tumors and blood-related cancer, or a combination thereof.

Other aspects of the invention provide methods for determining or predicting likelihood of recurrence of cancer in a patient. Such methods typically comprise measuring the amount of cells having active caspase 3 in a cell sample obtained from the patient. Generally, the amount of cells with active caspase 3 is measured from the cell sample comprising a tumor or cancer cell. Typically, the cell sample is a cancer biopsy sample. Any methods known in the art or methods that are developed after disclosure of the present invention for measuring the level of active caspase 3 can be used. For example, the level of active caspase 3 can be measured by cell staining (e.g., using an antibody specific for caspase 3) or measuring the level of active caspase 3 indirectly (e.g., by measuring one or more cleavage product of caspase 3 such as cytokeratin 18). Cell staining methods can include measuring radiolabeled complex or fluorescence labeled complex as well as other detection methods known to one skilled in the art. In one particular embodiment, methods of the invention include determining the amount of cells having active caspase 3 from a cell sample obtained from the patient's tissue or organ prior to a cancer treatment process. By determining the amount of cells having caspase 3 activity prior to starting a cancer treatment therapy using a cell sample obtained from the patient's tissue or organ being treated for the cancer, one can provide prognosis or determine the likelihood of cancer recurrence or relapse or select a proper cancer treatment protocol.

In some embodiments, the level of cells having caspase 3 activity is determined by a cell staining method. In one particular embodiment of the invention, when the amount or fraction of cells that stain positive for caspase 3 in the sample tested is 5% or higher, typically 10% or higher, and often 15% or higher, it is an indication of a likelihood of recurrence of cancer in the patient. In general, it has been found that if the sample cells contain at least 5% of cells, typically at least 10% cells, and often at least 15% cells having caspase 3, then the likelihood of cancer recurrence or relapse after cancer treatment therapy is at least 70%, typically at least 80%, and often at least 90%. Generally, a cancer or tumor biopsy sample is used to analyze the level of active caspase 3.

Other aspects of the invention include methods for reducing the probability or the likelihood or cancer recurrence in a patient undergoing a cancer treatment by administering a therapeutically effective amount of a caspase 3 inhibitor. In some embodiments, the caspase 3 inhibitor comprises Ac-DEVD-CHO (available from, e.g., Promega, Madison, Wis.); 5-[(S)-(+)-2-(Methoxymethyppyrrolidino]sulfonylisatin; Ac-DNLD-CHO (available from, e.g., EMD Biosciences, Inc.); Z-D(OMe)E(Ome)VD(OMe)-FMK (available from, e.g., A.G. Scientific, Inc.); Z-D(OMe)E(Ome)VD(OMe)-FMK; Ac-DMQD-CHO; Z-D(OMe)QMD(OMe)-FMK; 2-(4-Methyl-8-(morpholin-4-ylsulfonyl)-1,3-dioxo-1,3-dihydro-2H-pyrrolo[3,4-c]quinolin-2-yl)ethyl; acetate-Z-VAD-FMK; Ac-VAD-CHO; Boc-D-FMK, or a mixture thereof. It should be appreciated that the caspase 3 inhibitor can be administered with a cancer treatment or can be administered separately. Alternatively, caspase 3 inhibitor can be administered after completion of cancer treatment.

Another aspect of the invention provides a method for treating cancer in a patient by determining a need to administer a caspase 3 inhibitor by analyzing the caspase 3 activity from a cell sample obtained from the patient; administering a therapeutically effective amount of caspase 3 inhibitor to the patient if the level of cells with caspase 3 is at least 5%, typically at least 10%, and often at least 15%; and subjecting the patient to a cancer treatment therapy. The caspase 3 inhibitor can be co-administered with the cancer treatment therapy or it can be administered separately, e.g., prior to or after the cancer treatment therapy.

### DETAILED DESCRIPTION OF THE INVENTION

While early detection and treatment of cancer can increase survivability of a cancer patient, there are some patients that experience cancer recurrence or relapse even after early detection and treatment. Currently, there is no test that can determine which patient will experience relapse or recurrence of cancer after treatment.

Surprisingly and unexpectedly, the present inventors have discovered that the level of caspases in tumor cells can be used to determine whether a patient will likely experience relapse or recurrence of cancer. Furthermore, the level of caspases can also be used to determine the likelihood of survivability of the patient after cancer treatment. For example, one can determine the level of active caspases (e.g., caspases 3) from the patient's sample (e.g., patient's fluid sample—such as blood or serum—or even tumor cell sample that is obtained during a biopsy) prior to beginning a cancer treatment therapy (e.g., chemotherapy and/or radiotheraphy). If the level of cells with active caspase is at least 5%, typically at least 10%, and often at least 15%, then recurrence of cancer in the patient is likely to occur. Moreover, the survivability of the patient is significantly lower when the level of cells with active caspase is at least 5%, typically at least 10%, and often at least 15%.

The level of active caspase 3 can be determined by any of the methods known to one skilled in the art as well as methods that may be developed after the disclosure of the present application. For example, one can measure the levels of active caspase 3 in the patient's blood or serum sample by measuring the cleavage products of caspase 3 in the blood sample such as cleaved cytokeratin 18, which can be measured with ELISA, e.g., using M30 Apoptosense ELISA available from Peviva AB (Bromma, Sweden). See www.peviva.se.

Caspases are a family of cysteine proteases that play essential roles in various cell functions. Some of the most important roles of caspases are believed to be their role in apoptosis, necrosis, and inflammation. While some of the functions of caspases have been identified, there are still a wide variety of unknown functions of caspases. Some aspects of the invention are based on a discovery by the present inventors during experiments to identify other roles of caspases. In particular, experiments were performed by the present inventors to identify other roles of caspases that can be used in various diagnostic and/or therapeutic purposes.

In cancer treatment, apoptosis is a well-recognized cell death mechanism through which cytotoxic agents (e.g., anti-cancer drugs, radiation, etc.) kill tumor cells. The present inventors have previously discovered that dying tumor cells use the apoptotic process to generate potent growth-stimulating signals to stimulate the repopulation of tumors undergoing radiotherapy. Surprisingly and unexpectedly, the present inventors have discovered that activated caspase 3, a key executioner of apoptosis, also plays key roles in the growth stimulation. One downstream effector that caspase 3 regulates is prostaglandin E₂ (PGE₂), which can potently stimulate growth of surviving tumor cells. The present inventors have discovered deficiency of caspase 3 either in tumor cells or in tumor stroma caused significant tumor sensitivity to radiotherapy in xenograft or mouse tumors. The present inventors have also discovered that higher levels of activated caspase 3 in tumor tissues were correlated with significantly increased rate of recurrence and deaths in cancer patients.

There is a massive amount of cell death during cytotoxic cancer therapy such as radiation therapy. It is normally assumed that dying or dead cells get absorbed by “scavenger” cells such as macrophages or other surviving cells in the vicinity. The small number of surviving tumor cells, if any, would then gradually and slowly proliferate and re-establish the tumor. However, studies by others have indicated that tumors respond to radiotherapy by initiating a process called “accelerated repopulation.” In this process, the few surviving cells that escaped death after exposure to radiotherapy or chemotherapy rapidly repopulate the badly damaged tumor by proliferating at an significantly accelerated pace. This phenomenon, for which little is understood at the molecular level, has played an important role in modern radiotherapy and chemotherapy.

Great efforts have been made in the past to understand the molecular mechanism of tumor repopulation after cytotoxic therapy. For example, some have shown that radiation-induced up-regulation of angiogenic activities in tumors has been linked with activation of upstream transcriptional factors such as HIF-1. Recent studies have also indicated the importance of macrophages in facilitating tumor recovery after radiation. Furthermore, the integrity of endothelial cells has been implicated in tumor response to radiotherapy. These reports, while revealing interesting mechanisms of tumor re-growth, fall short in describing the initial driving events responsible for tumor repopulation after radiotherapy.

As disclosed in the present inventors' related U.S. patent application Ser. No. 12/985,324, filed Jan. 5, 2011, which is incorporated herein by reference in its entirety, the present inventors have discovered that dying cells in the tumor mass provide the initial signals to promote tumor repopulation. Specifically, the present inventors have discovered that dying cells release growth-promoting signals to stimulate the proliferation of surviving cells. In the related U.S. Patent Application, which has been incorporated by reference supra, the present inventors have shown that intratumoral dying cells promote rapid repopulation of tumors from a small number of live tumor cells. In addition, as shown in the Examples section below, the present inventors have discovered that caspase 3, a cysteine protease involved in the “execution” phase of cellular apoptosis, is a key regulator of growth-promoting signals generated from the dying cells. Furthermore, the present inventors have disclosed various cancer biology and cancer therapy using such a discovery.

Determining the level of activated caspase 3 can be readily achieved by using any of the direct or indirect methods including, but not limited to, staining a tumor or cancer cell sample as described in the Examples section below, by measuring the level of cleavage product of caspase 3 such as cytokeratin 18, as well as other methods known to one skilled in the art. It should be appreciated that one can measure the level of activated caspase 3 using a various labeling techniques such as fluorescence, radio active labeling, phosphorescence, etc. In some embodiments, if the level of cells having caspase 3 activity is at least 5%, typically at least 10%, and often at least 15% in the cell sample (e.g., tumor biopsy sample), there is a likelihood of cancer or tumor recurrence or relapse from a cancer treatment therapy.

Other aspects of the invention provide methods for treating cancer in a patient. Such methods include administering cancer treatment to the patient and administering a therapeutically effective amount of caspase 3 inhibitor to the patient. The caspase 3 inhibitor can be administered prior to or after administering the cancer treatment to the patient. The caspase 3 inhibitor can also be co-administered with the cancer treatment therapy. Typically, the cancer treatment includes chemotherapy, radiation therapy, or a combination thereof. Often the caspase 3 inhibitor is administered 24 hours or less, typically 5 hours or less, often 1 hour or less prior to administering a cancer treatment. In other embodiments, the caspase 3 inhibitor is administered 1 hour or more, typically 5 hours or more, often 24 hours or more after administering cancer treatment.

In other aspects of the invention, one can determine a cancer treatment therapy protocol by analyzing or determining the amount of cell with caspase 3 in the cell sample. Typically, the cell sample comprises or consists essentially of a cancer or tumor biopsy sample. For example, if the level or the amount of cells with caspase 3 in the cell sample is 5% or more, typically 10% or more, and often 15% or more, then the cancer treatment therapy protocol will include administering a therapeutically effective amount of a caspase 3 inhibitor along with the conventional cancer treatment. If, however, the level or the amount of cells with caspase 3 is less than the amount disclosed herein, administration of a caspase 3 inhibitor is generally not necessary.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Human Tumor Samples

Clinical human tumor samples from Princess Margaret Hospital and Shanghai No. 1 People's Hospital were acquired with informed consent following protocols approved by institutional review boards at the two hospitals.

### Cells and Tissue Culture Conditions

A variety of cancer and fibroblast cells were used in this study. Among these are the mouse breast cancer cell line 4T1, mouse fibroblast cell lines NIH3T3, human cancer cell lines MCF-7 (breast cancer line), MDA-MB231 (breast cancer line), HCT116 (colon cancer line), and human fibroblast cell strain IMR-90. All the above murine and human cancer lines are available from American Type Tissue Culture (ATCC, Manassas, Va., USA). In addition, wild type and caspase-deficient mouse embryonic fibroblast cells were obtained from Dr. Richard Flavell of Yale University (New Haven, Conn.). For maintenance of the cells, Dulbecco's Eagles's Medium (DMEM) (Invitrogen, Carlsbad, Calif.) supplemented with 10% fetal bovine serum was used to culture the cells.

### Bioluminescence Imaging

For imaging luciferase, the IVIS200 instrument from Caliper Life Sciences (Hopkinton, Mass.) was used. For tissue cultured cells, luciferase signal was imaged by adding PBS or colorless OptiMEM medium (Invitrogen, Carlsbad, Calif.) with D-luciferin (Caliper Life Sciences, Hopkinton, Mass.) at a concentration of 0.15 mg/mL. The cells were imaged at a set time point (e.g., 10 minutes) after the administration of D-luciferin in order to ensure comparability of signals from different samples. After images were taken, manufacturer supplied software was used to process the images for quantitative data.

To monitor growth of Fluc-labeled cells in vitro, about 500 or 1000 cells were mixed together with an overwhelming number of (for example 2×10⁵) unlabeled cells either right after irradiation or 24 hrs after irradiation (or other cytotoxic treatments) the cells were then monitored at different times for up to 8 days after seeding by use of IVIS200.

Growth of Fluc-labeled tumor cells in vivo was followed through non-invasive bioluminescence imaging using the IVIS200 instrument (Caliper Life Sciences, Hopkinton, Mass). Mice to be imaged were injected with 150 mg/kg of D-luciferin (obtained from Caliper Life Sciences) intraperitoneally in 200 μl of PBS and then anesthetized with continuous flow of isofluorane. Imaging of the mice was carried out 10 minutes later. The time between injection and imaging were kept constant among different batches of mice.

### Monitorin Tumor Cell Growth in Mice

Different approaches were used to establish tumor growth than traditionally adopted by other studies. In most of the studies by the present inventors, a very small number of Fluc-labeled tumor cells (500-1000 cells each) was injected either alone or together with other unlabeled cells (either tumor cells or fibroblast cells) that are either irradiated or un-irradiated. Tumor growth from these cells was monitored through the quantification of bioluminescence signals emitted from labeled tumor cells using IVIS200 instrument following manufacturer's instructions. In some cases, 500-1000 Fluc labeled tumor cells were injected into (7-9 mm in diameter) tumors that have been established from unlabeled tumor cells.

### Tumor Growth Delay Experiments

To evaluate the function of caspase 3 in tumor radiotherapy, about 3×10⁶ MCF-7 and MCF-7CASP3 tumor cells were injected subcutaneously with matrigel were established in female nude mice implanted with estrogen pellets (1.7 mg/pellet, 60 day release formula, Innovative Research of American). When tumors reach 5-7 mm in diameter, they were exposed to x-irradiation (2×6 Gy). The sizes of the tumors were measured every other day with a caliper. In separate experiments, B16F10 melanoma cells were injected into syngeneic C57BL/6 wild type and caspase 3 knockout (−/−) mice (Jackson Laboratory) and tumor growth experiments were conducted when tumor reach 5-7 mm in diameter.

### Gene Transduction Into the Cells

To transduce various exogenous genes into target cells, main vehicle used was the lentivirus vector. Mostly the pLEX system was used, which is a lentivector system purchased from Open Biosystems (Huntsville, Ala.). Genes that were cloned into this vector include: the firefly luciferase gene (Fluc) obtained commercially from Promega (Madison, Wis.); a truncated, activated iPLA₂ gene obtained from RT-PCR. Lentiviral vectors encoding shRNAs against caspase 3 or iPLA₂ were also used. All the lentiviral vectors were packaged into live lentiviral viruses in 293T cells following manufacturer's instructions.

### Irradiation of Cells and Mice

X-ray irradiation of cells and mice were carried out in a RS-2000 Biological Irradiator purchased from Rad Source Corporation (Atlanta, Ga.). The dose rate for the machine was about 1 Gy/minute.

### Measurement of Arachidonic Acid Release

To measure arachidonic release, cells were plated in 6-well dishes at a density of 1.0-2.0×10⁵ cells/well. About 1.0 μCi of [³H]-arachidonic acid (obtained commercially from GE Healthcare Life Sciences) was then added to the cells, which has about 1 ml of DMEM medium that was serum free and with 0.5 mg/ml of lipid-free bovine serum albumin (Sigma Chemical Co., St Louis, USA). After 16 h, the cells were washed with fresh medium 3× and incubated with 3 ml of DMEM medium supplemented with 5% serum. After another 5 h, when arachidonic acid in the supernatant reached a steady state level, the cells were exposed to 8-10 Gy of x-rays. Supernatants were then removed at 4, 24, and 48 h from the cells and counted with a scintillation counter for quantification of [³H]-arachidonic acid.

### ELISA Measurement of PGE₂

To evaluate the PGE₂ secretion from cells, about 2×10⁵ cells/well were plated in 6-well dishes. Cells were cultured in DMEM medium supplemented with 2% fetal bovine serum. They were then irradiated with x-rays (8-12 Gy). Supernatant from the cells were taken right before and 24 hrs after cellular irradiation. PGE₂ in the supernatants were then measured by use of an ELISA kit purchased commercially from R&D Systems (Minneapolis, Minn., USA).

### Molecular Cloning

The pLEX lentiviral vectors system was used to deliver reporter and other genes into target cells. The system was obtained commercially from Open Biosystems (Huntsville, Alabama, USA). The genes transduced through pLEX include: 1) firefly luciferase gene, which was transferred from the plasmid pGL4.31-luc2 from Promega (Madison, Wis.); 2) a truncated version of the mouse calcium-independent phospholipase A₂, which was amplified through RT-PCR from murine mRNA by using the primers disclosed in the previously incorporated U.S. Provisional Patent Application No. 61/499,094, filed Jun. 20, 2011. The Pfx polymerase (Invitrogen, Carlsbad, Calif.) was used for the PCR amplification. The amplified fragment encodes aa453-679 (which is equivalent to aa514-733 of human iPLA2) of murine iPLA₂ (accession# NM-016915), which has been shown to be a constitutively active caspase cleavage product. The fragment was cloned into PCR-Blunt and excised with Spe I and Not I restriction enzymes and cloned into the Spe I and Not I sites of a modified pLEX plasmid. The modified pLEX plasmid has an influenza hemagglutinin (HA) tag inserted between Not I and Mlu I (two of the unique restriction sites in pLEX) so that genes inserted into the Not I site can be fused with the HA tag. In addition, lentiviral vectors encoding shRNA-encoding minigenes targeted against the murine iPLA₂ gene was obtained from Open Biosystems (Huntsville, Ala.). These genes were carried in the pLKO.1 lentiviral vectors system. The one that showed most efficacy had the targeting sequence disclosed in U.S. Provisional Patent Application No. 61/499,094, i.e., catalog #Rmmu534-NM-016915 from Open Biosystems (Huntsville, Ala.).

For knocking down the PGE2 receptor EP2, a ready made vector from Open Biosystems (catalog #RMM3981-9594402) was used. The target sequence for this shRNA is also disclosed in U.S. Provisional Patent Application No. 61/499,094.

A caspase 3 targeted, shRNA minigene-encoding lentiviral vector was also constructed. The sequence of this shRNA is also disclosed in U.S. Provisional Patent Application No. 61/499,094. A double stranded oligo containing the above shRNA sequence was cloned into the pLVTHM vectors, which is a known lentiviral vector.

For dominant caspase 3, a key cystein in the catalytic domain of wild type murine caspase 3 was mutated (C163→A) through site directed mutagenesis. The mutant version was then cloned into the lentiviral vector pLEX. In all cases, manufacturer's instructions or published protocols were followed to produce live, replication-deficient recombinant lentiviral vectors in 293T cells.

### Western Blot Analysis

Cellular lysates were obtained under most circumstances using the standard RIPA buffer. For running western blot analysis, 40-60 μg/per sample was used for gel electrophoresis. Samples were usually heated to 95° C. for 5 minutes before loading into electrophoresis gel.

For cytochrome c western analysis, a digitonin-based buffer was used to lyse the cells. The buffer consists of: 190 μg/ml of digitonin (obtained from Sigma Chemical, St Louis, Mo.), 75 mM NaCl, 1 mM NaH₂PO₄, 8 mM Na₂HPO₄, 250 mM sucrose. Treatment of cells with this buffer leads to plasma membrane leakage of cytosolic material. After a brief spin, the supernatant were used to analyze for cytochrome c leakage into the cytosol through western blot analysis.

### Chemicals Used in This Study

The pan caspase inhibitor z-VAD-fmk, was obtained from EMD Bisciences (Gibbstown, NJ.). Cyclooxygenase inhibitor indomethacin and a long half-life version of PGE₂, 16,16-dimethyl PGE₂ (dmPGE₂) were both obtained from Caymen Chemical (Ann Arbor, Mich.).

### Immunofluorescence and Immunohistochemistry Analysis

For immunofluorescence analyses of the following proteins: caspase 3, SMA, GFP, antibodies were used. Paraffin-embedded tumor tissues were used. For F4/80, sectioned frozen tumor tissues were used because of antibody functionality.

### IHC Analysis of Human Cancer Samples

Tumor tissue samples from two cohorts of patients were used to determine the correlation between cleaved caspase 3 levels and clinical outcomes. In the first cohort, 39 tumor samples from patients with head and neck squamous cell carcinoma (HNSCC) treated at the Princess Margaret Hospital, Toronto, Canada were examined. Patient characteristics are provided in the following table:

| Clinical | | |
|---|---|---|
| characteristics of the HNSCC patients | | Number (%) |
| Age (years) | ≦65 | 19 (49%) |
| | >65 | 20 (51%) |
| Sex | Male | 30 (77%) |
| | Female | 9 (23%) |
| Tumor site | Larynx | 27 (69%) |
| | Hypopharynx | 12 (31%) |
| Tumor stage | III | 14 (36%) |
| | IVA | 24 (61%) |
| | IVB | 1 (3%) |
| Relapse | Local/Regional | 15 (39%) |
| | Distant | 8 (21%) |
| Treatment | Conventional conformal RT | 36 (92%) |
| | IMRT | 3 (8%) |
| | RT alone | 26 (67%) |
| | CRT (70 Gy/35 f/7 weeks) | 13 (33%) |

RT: radiotherapy
IMRT: intensity-modulated radiotherapy
CRT: concurrent chemo-radiation

In the second cohort, 48 tumor samples from patients with advanced breast cancer patients treated at Shanghai No. 1 People's Hospital were examined. Patient characteristics are provided in the following table:

| Clinical characteristics of breast cancer patients. | | Number (%) |
|---|---|---|
| Age (years) | ≦50 | 21 (43.8%) |
| | >50 | 27 (56.2%) |
| Menopause* | Pre | 26 (54.2%) |
| | Post | 22 (45.8%) |
| Tumor type | Infiltrating ductal carcinoma | 44 (91.7%) |
| | Infiltrating lobular cancinoma | 4 (8.3%) |
| Tumor stage | I | 3 (6.2%) |
| | IIa | 13 (27%) |
| | IIb | 13 (27%) |
| | IIIa | 8 (16.6%) |
| | IIIb | 1 (2%) |
| | IIIc | 10 (20.8) |
| Molecular pathology | ER positive | 27 (56.2%) |
| | PR positive | 25 (52%) |
| | Her-2 positve | 11 (23%) |
| Treatment | Chemotherapy | 44 (91.6%) |
| | Tamoxifen | 28 (58.3%) |
| | Radiotherapy | 23 (47.9%) |
| Metastasis | | 18 (37.5%) |
| Death | | 15 (31.2%) |

*all are female breast cancer patients who have undergone surgery
ER: estrogen receptor
PR: Progesterone receptor

### Protocol for Staining Activated Caspase 3 in Paraffin-Embedded Tumor Samples

The following general procedures were used to stain and determine the level of activated caspase 3 activity from tumor cell samples.

The following solutions were prepared with Milli-Q or equivalently purified water. (1) Wash Buffer: 1×TBS/0.1% Tween-20 (1×TBST): To prepare 1 L, add 100 ml 10×TBS to 900 ml of deionized water (dH₂O). Add 1 ml Tween-20 and mix. 10× Tris Buffered Saline (TBS): To prepare 1L, add 24.2 g Trizma® base (C₄H₁₁NO₃) and 80 g sodium chloride (NaCl) to 1 L dH₂O. Adjust pH to 7.6 with concentrated HCl. (2) Antibody Diluent: SignalStain® Antibody Diluent #8112 is commercially available. http://www.cellsignal.com/products/8112.html. TBST/5% normal goat serum: To 5 ml 1×TBST, add 250 μl normal goat serum (http://www.cellsignal.com/products/5425.html). PBST/5% normal goat serum: To 5 ml 1×PBST, add 250 μl normal goat serum. 1×PBS/0.1% Tween-20 (1×PBST): To prepare 1L, add 100 ml 10×PBS to 900 ml dH2O. Add 1 ml Tween-20 and mix. 10× Phosphate Buffered Saline (PBS): To prepare 1 L, add 80 g sodium chloride (NaCl), 2 g potassium chloride (KCl), 14.4 g sodium phosphate, dibasic (Na₂HPO₄) and 2.4 g potassium phosphate, monobasic (KH₂PO₄) to 1 L dH₂O. Adjust pH to 7.4. (3) Antigen Unmasking: Citrate: 10 mM Sodium Citrate Buffer: To prepare 1 L, add 2.94 g sodium citrate trisodium salt dihydrate (C₆H₅Na₃O₇.2H₂O) to 1 L dH₂O. Adjust pH to 6.0. EDTA: 1 mM EDTA: To prepare 1 L, add 0.372 g EDTA (C₁₀H₁₄N₂O₈Na₂. 2H₂O) to 1 L dH₂O. Adjust pH to 8.0. TE: 10 mM Tris/1 mM EDTA, pH 9.0: To prepare 1 L, add 1.21 g Trizma® base (C₄H₁₁NO₃) and 0.372 g EDTA (C₁₀H₁₄N₂O₈Na₂.2H₂O) to 950 ml dH₂O. Adjust pH to 9.0, then adjust final volume to 1 L with dH₂O. Pepsin: 1 mg/ml in Tris-HCl, pH 2.0. (4) 3% Hydrogen Peroxide: To prepare, add 10 ml 30% H₂O₂ to 90 ml dH₂O. (5) Blocking Solution: TBST/5% normal goat serum: to 5 ml 1×TBST, add 250 μl normal goat serum. (6) Detection System: Use according to manufacturer's recommendations. http://www.cellsignal.com/support/protocols/IHC-Paraffin-SignalStain.html. (7) DAB Reagent or suitable substrate: Prepared according to manufacturer's recommendations.

Deparaffinization/Rehydration of cell samples were performed as follows while keeping the slides wet throughout the process. (1) Deparaffinize/hydrate sections: (a) Incubate sections in three washes of xylene for 5 minutes each. (b) Incubate sections in two washes of 100% ethanol for 10 minutes each.; and (c) Incubate sections in two washes of 95% ethanol for 10 minutes each. (2) Wash sections twice in dH₂O for 5 minutes each.

Antigen Unmasking was performed by consulting product datasheet for specific recommendation for the unmasking solution. For example, (1) For Citrate: Bring slides to a boil in 10 mM sodium citrate buffer, pH 6.0; maintain at a sub-boiling temperature for 10 minutes. Cool slides on bench top for 30 minutes. (2) For EDTA: Bring slides to a boil in 1 mM EDTA, pH 8.0: follow with 15 minutes at a sub-boiling temperature. No cooling is necessary. (3) For TE: Bring slides to a boil in 10 mM TE/1 mM EDTA, pH 9.0: then maintain at a sub-boiling temperature for 18 minutes. Cool at room temperature for 30 minutes. (4) For Pepsin: Digest for 10 minutes at 37° C.

Staining was performed by consulting product datasheet for recommended antibody diluent. Briefly, (1) Wash sections in dH₂O three times for 5 minutes each. (2) Incubate sections in 3% hydrogen peroxide for 10 minutes. (3) Wash sections in dH₂O twice for 5 minutes each. (4) Wash sections in wash buffer for 5 minutes. (5) Block each section with 100-400 μl blocking solution for 1 hour at room temperature. (6) Remove blocking solution and add 100-400 μl primary antibody diluted in recommended antibody diluent to each section*. Incubate overnight at 4° C. (7) Equilibrate SignalStain® Boost Detection Reagent to room temperature. (8) Remove antibody solution and wash sections in wash buffer three times for 5 minutes each. (9) Cover section with 1-3 drops SignalStain® Boost Detection Reagent as needed. Incubate in a humidified chamber for 30 minutes at room temperature. (10) Wash sections three times with wash buffer for 5 minutes each. (11) Add 100-400 μl DAB or suitable substrate to each section and monitor staining closely. (12) As soon as the sections develop, immerse slides in dH₂O. (13) If desired, counterstain sections in hematoxylin per manufacturer's instructions. (14) Wash sections in dH₂O two times for 5 minutes each. (15) Dehydrate sections as follows: (a) Incubate sections in 95% ethanol two times for 10 seconds each. (b) Repeat in 100% ethanol, incubating sections two times for 10 seconds each. (c) Repeat in xylene, incubating sections two times for 10 seconds each. (16) Mount coverslips.; and (17) observe slide.

### RESULTS

### Tumor Cell Repopulation Stimulated by Cell Death in Vitro

Experiments were carried out to examine if dying tumor cells could indeed stimulate the growth of living tumor cells. In order to simulate in vivo scenarios where the vast majority of tumor cells are killed by radiation or chemotherapy, a small number (about 500) of firefly luciferase (Fluc)-labeled murine breast cancer 4T1 cells were seeded onto a bed of a much larger number (2.5×10⁵) of unlabeled “feeder” 4T1 tumor cells that were irradiated with x-rays at different doses. Growth of the small number of labeled living cells was then monitored through non-invasive bioluminescence imaging. Results indicated that 4T1Fluc cells grew significantly faster when seeded onto dying cells than when seeded alone. In addition, there was a dose-dependent response from the feeder cells, with non-irradiated feeder cells exhibited no supportive roles and those irradiated with higher radiation doses exhibiting higher growth-enhancing ability. Additional supporting evidence came from combinations of other dying v. living cell types, which also showed growth-stimulating properties.

Because in solid tumors stromal cells play important roles in modulating tumor growth, whether dying fibroblast cells could promote tumor cell growth was also evaluated. Lethally irradiated mouse embryonic fibroblast cells stimulated the growth of different Fluc-labeled tumor cells significantly in vitro.

### Tumor Cell Repopulation Stimulated by Cell Death in Vivo

Whether cell death-stimulated tumor cell proliferation could be observed in vivo was also examined. A mix of untreated, Fluc-labeled and lethally irradiated, unlabeled tumor cells (at a ratio of 1:250, or 1000 live 4T1Fluc cells mixed with 2.5×10⁵ unlabeled, lethally irradiated 4T1 cells) were injected subcutaneously into the hind legs of nude mice. Subsequently the growth of the Fluc-labeled tumor cells was followed non-invasively over time through bioluminescence imaging. As controls, an equal number of Fluc-labeled 4T1 tumor cells mixed with live, unlabeled 4T1 tumor cells were injected into contra-lateral hind legs. Results showed that the presence of lethally irradiated tumor cells significantly increased the growth of Fluc-labeled tumor cells when compared with Fluc-labeled tumor cells injected together with live tumor cells. In fact the difference in the intensities of luciferase signals between the two groups grew exponentially larger and reached as great as 700 fold at the end of the experiment.

Similar in vivo tumor growth-promoting properties were also observed for mouse embryonic fibroblasts (MEF) that were irradiated. Fluc-labeled 4T1 co-injected with irradiated fibroblast cells grew to signal intensities 400 fold more than those from 4T1-Fluc cells injected alone in contra-lateral hind legs.

### Caspase 3 Regulates Tumor Cell Repopulation in Vitro

Surprisingly and unexpected, it was discovered that caspases, the proteases that are involved in both the initiation and the execution of programmed cell death, are also involved in regulating the growth-promoting properties of dying cells. For example, experiments using mouse embryonic fibroblast (MEF) cells with genetic deletion of their Caspase 3 gene and evaluating them for the ability of these cells to support the growth of a small number of Fluc-labeled tumor cells showed that deficiencies in Casp3 significantly compromised the ability of lethally irradiated MEF cells to stimulate the growth of Fluc-labeled murine (4T1) and human (MDA-MB231 and HCT116) tumor cells. The proliferation of Fluc-labeled tumor cells among the irradiated Caspase 3 deficient (Casp3^{-/-}) cells was close to Fluc-labeled tumor cells seeded alone, indicating that caspase 3 was largely responsible for growth stimulation by dying cells.

The importance of caspase 3 was also confirmed in lethally irradiated 4T1 cells through shRNA-mediated knockdown of Casp3 expression in feeder cells. It was similarly confirmed in the human breast cancer cell line MCF7, which is deficient in casp3 expression. Exogenous expression of caspase 3 significantly increased the ability of lethally irradiated MCF-7 cells to promote co-seeded MCF-7Fluc cells.

Because Casp3^{-/-} and wild type MEF cells showed similar clonogenic survival after irradiation, it is believed that the observed defect for Casp3^{-/-} MEF cells was not due to the lack of cell death in these cells after radiation. Further data were obtained indicating how caspase 3 status affects the modes of cell death in 4T1, MEF, and MCF-7 after radiation.

To examine whether the proteolytic activity of caspase 3 is required, a dominant-negative version of caspase 3 (C163A) was transduced to inhibit caspase 3 cleavage activity in 4T1 cells. Data indicated that 4T1 cells transduced with a dominant-negative Casp3 (C163A) gene completely lost its ability to support the growth of 4T1Fluc cells. Similar results were also obtained by use of a chemical inhibitor of caspase 3 z-VAD-fmk.

To confirm that caspase 3 was activated in irradiated cells, comprehensive immunoblot analyses of various proteins in the apoptotic pathway in irradiated 4T1, and MEF cells were carried out. Data indicated caspases 3 and 9 and downstream cytochrome c were activated in both 4T1 and MEF cells in a dose-dependent manner while caspase 8 was not significantly activated.

### Caspase 3 Regulation of Tumor Cell Repopulation in Vivo

To examine the role of caspase 3 in cell-death stimulated tumor repopulation in vivo, lethally irradiated MEF cells with different caspase 3 status were mixed together with a small number (about 500) of Fluc-labeled 4T1 cells and injected subcutaneously into nude mice. In contrast to potent stimulation of 4T 1 Fluc tumor cellular growth by lethally irradiated wild type MEF cells, significantly attenuated growth stimulation was observed for lethally irradiated caspase 3-deficient MEF cells. The difference between the two groups was as great as 1000 fold_at later stages of observation. In fact, in separate experiments, 4T1-Fluc cells injected together with lethally irradiated caspase 3-deficient MEF cells grew at a similar rate as those 4T1Fluc cells injected alone in the contra-lateral legs, indicating a lack of growth stimulation from Casp3^{-/-} cells.

The importance of caspase 3 in regulating growth-promoting properties of dying cells in vivo was also confirmed by co-injecting 4T1-Fluc cells with lethally irradiated 4T 1 transduced with an shRNA minigene targeted against caspase 3. A significant reduction in the ability of lethally irradiated 4T1 cells to stimulate the growth of 4T1-Fluc cells were observed, consistent with the results obtained with Casp3^{-/-} MEF cells.

### Caspase 3 Activation and Growth of Injected Tumor Cells in Established Tumors

To confirm that caspase 3 activation is indeed activated in solid tumors during radiotherapy, murine 4T1 tumor cells transduced with a novel luciferase/GFP and proteasome-based reporter were established. Tumors were then irradiated with x-rays (6 Gy) when they reached 5-7 mm in diameter and observed for bioluminescence signals periodically afterwards. Data clearly showed that caspase 3 was significantly activated (as much as 30 fold at its peak) at days 3, 5, and 7 after radiotherapy.

In another experiment, a small number of Fluc-labeled 4T1 tumor cells (about 1000) were injected into irradiated and non-irradiated tumors in the same group of mice and observed for growth. Results demonstrated that cells injected into irradiated tumors grew at a significantly faster rate than those injected into the non-treated tumors, consistent with earlier results.

In another experiment, 4T1 cells stably transduced with green fluorescence protein (GFP) was injected into irradiated tumors and allowed to grow for 5-8 days. The mice were then sacrificed and tumors excised and examined for expression of various proteins. Data showed a clear relationship between activated caspase 3 and the proliferation of injected, GFP-labeled tumor cells in irradiated tumor cells, as demonstrated by the complementary pattern of caspase 3 staining and injected tumor cell staining In contrast, very little injected tumor cell growth and caspase activation was observed in non-irradiate tumors. In addition to caspase activation, irradiated tumors also showed increased, localized neovaculature.

### Caspase 3 Activates iPLA₂ to Stimulate Tumor Repopulation

Attempt was made to identify the downstream factors of caspase 3 that were involved in generating growth-promoting factors from the dying cells. Calcium independent phospholipase A₂ (iPLA₂) was examined because it is known that its phosopholipase activity is activated by caspase 3 cleavage. In addition, the activation of iPLA₂ was shown to increase production of arachidonic acid (AA), whose downstream eicosanoid derivatives (i.e., prostaglandin E₂), had been implicated in stimulating tumor growth and stem cell proliferation. To evaluate the potential involvement of caspase-activated iPLA₂-AA-PGE₂ axis in cell death-induced tumor cell proliferation, shRNA against the iPla₂ gene was transduced into 4T1 tumor cells or wild type MEF cells and examined whether these cells, when lethally irradiated, could still support Fluc-labeled tumor cell growth as much as their wild-type counterpart. Data showed that in both MEF and 4T1 cells, expression of shiPLA₂ significantly reduced ability of these cells to stimulate Fluc-labeled 4T1 cellular proliferation. Western blot data also showed that iPLA₂ was activated in a caspase 3-dependent manner in both 4T1 and MEF cells.

The importance of caspase 3-mediated activation of iPLA₂ was further confirmed when a truncated version of the iPla₂ gene (ΔiPla₂), which encoded an iPla₂ fragment (containing the catalytic domain) that was a predicted product of caspase 3 cleavage of iPla₂, was transduced into caspase 3-deficient MEF cells. After ΔiPla₂ transduction, lethally irradiated Casp3^{-/-} cells had significantly increased ability to stimulate the growth of Fluc-labeled 4T1-Fluc cells both in vitro and in vivo when compared with parental Casp3^{-/-} cells.

Knocking down iPla₂ gene expression showed significant reduction in the ability of lethally irradiated MEF cells to stimulate the growth of 4T1-Fluc cells in vivo. Furthermore, by use of established wild type or iPla₂ knockdown 4T1 tumors, it was shown that growth rate of subsequently transplanted 4T1 cells was significantly reduced.

### Caspase 3 and iPLA₂ Stimulate AA and PGE₂ Release

Experiments were also conducted to confirm the role of caspase 3 in regulating the activity of the iPLA₂ by examining its enzymatic activities. Because arachidonic acid is one of two main catalytic products of activated iPLA₂ (the other being lysophosphatidic choline, or LPC), radiation-induced release of arachidonic acid into the extracellular milieu was measured to examine potential relationship between caspase 3 and arachidonic acid release. Results showed that radiation stimulated the release of arachidonic acid into the supernatant significantly in wild type MEF cells. However, the release was significantly reduced in Casp3^{-/-} MEF cells, indicating a significant role for caspase 3. In addition, arachidonic acid (AA) release was also significantly reduced in 4T1 cells with effective knockdown of the Casp3 gene expression.

Because prostaglandin E₂ (PGE₂), a key regulator of tumor growth, is a downstream product of AA, PGE₂ production induced by ionizing radiation was measured in the supernatants of cells. Results indicated that exposure to ionizing radiation significantly induced the production of PGE₂ in wild type MEF cells as well as in 4T1 tumor cells. However, in Casp3^{-/-} MEF cells and in 4T1 cells transduced with a shRNA against caspase 3, the production of PGE₂ in irradiated cells was significantly reduced. In contrast, transduction of a constitutively active, truncated iPla₂ (which is equivalent to a caspase-cleaved version of iPLA₂), significantly restored PGE₂ production in caspase 3 deficient (Casp3^{-/-}) MEF cells.

In other experiments, cyclooxygenase 2 (Cox 2) and its upstream transcription factor NF-kB, which have crucial roles in PGE₂ production, were activated in a caspase 3-independent manner. Consistently, indomethacin (a Cox 1 &2 inhibitor) administration effectively suppressed growth of intratumorally injected 4T1 and HCT116 cells.

The importance of PGE₂ was demonstrated by the fact that treatment of a small number (about 1000) of tumor cells with PGE₂ gave the treated cells a significant head start, allowing them to grow at a faster pace than non-treated cells when injected into mice. On the other hand, shRNA mediated down-regulation of EP₂, a receptor for PGE₂, in 4T 1 Fluc cells significantly attenuated the proliferation of the latter when seeded together with lethally irradiated 4T1 cells.

### Caspase 3 and Radiosensitivities of Tumors in Mice and Man

To examine the prospect of enhancing cancer radiotherapy through inhibition of caspase 3, experiments in mouse tumor models were conducted.

In one experiment, MCF-7 cells, which are naturally deficient in caspase 3 expression were used. Tumor xenograft in female nude mice was established using both parental MCF-7 cells as well as a modified MCF-7 cell line with an exogenously transduced copy of caspase 3 gene. The transduction of casp 3 into parental MCF-7 cells rendered them significantly more susceptible to apoptosis. Surprisingly, caspase 3 transduction also enabled MCF-7 cells to form tumors at a faster pace. In addition, the presence of caspase 3 made MCF-7CASP3 tumors significantly more resistant to radiotherapy than parental MCF-7 tumors, which disappeared completely after 2×6 Gy or x-rays and did not re-grow during the entire course of observation. In contrast, MCF-7CASP3 kept growing after irradiation despite being slowed down. These surprising results indicated that deficiency of caspase 3 activity in tumor cells will render the whole tumor significantly more susceptible to radiotherapy.

In another experiment, the potential role of stroma-derived caspase 3 activities in tumor response to radiotherapy was examined because stroma accounted for a significant percentage of tumor mass. Tumors were established in wild type as well as Casp3^{-/-} mice with B16F10 cells, which is a metastatic and radioresistant tumor line syngeneic with the C57BL/6 mice. Radiotherapy (2×8 Gy) was carried out when tumor reached 5-7 mm in diameter. Results indicated that B16F10 tumor grown in Casp3^{-/-} mice exhibit significant sensitivity to radiotherapy when compared with those in wild type C57BL/6 mice. These results confirmed the involvement of caspase 3 in both tumor cells and tumor stroma in stimulating tumor response to radiotherapy.

Caspase 3 status in two cohorts of human cancer patients was also examined. In the first cohort, 39 head and neck cancer patients treated with radiotherapy, or chemo-radiotherapy at the Princess Margaret Hospital in Toronto, Canada were examined for activated caspase 3 through immunohistochemical analysis. Consistent with results in mice, patients with high cleaved (and thus activated) caspase 3 levels in their tumor samples showed significantly (p=0.011) higher rate of tumor recurrence. In the second cohort, 48 advanced stage breast cancer patients treated at Shanghai No. 1 People's hospital were analyzed for activated caspase 3 expression through IHC analysis. Results indicated that patients with high cleaved caspase 3 staining were at a significant (p=0.0002) disadvantage in terms of survival. Further supporting evidence came from analysis of public domain microarray data by the present inventors from a previously published study. In 249 breast cancer patients from Sweden and Singapore, elevated caspase 3 mRNA levels correlated with significantly elevated risk (p=0.0001) of relapse. These results show that elevated tumor caspase 3 levels predict for worse treatment outcomes in human cancer patients.

### DISCUSSION

For tumor cells exposed to severe stress such as cytotoxic cancer therapeutics, life and death are the two polar opposites of cellular fates. The present inventors have discovered that these two fates are inextricably associated, manifested by the surprising and unexpected observation that apoptotic tumor cells stimulate the repopulation of tumors from a small number of surviving cells. Even more surprising and unexpected is the revelation that caspase 3, the master “executioner” during apoptotic cell death, serves as a direct link between cell death and tumor repopulation. Despite the paradoxical nature of these results at first glance, however, the link between cellular death and proliferation is one of the key mechanisms of metazoan tissue homeostasis exploited by tumors to preserve themselves when damaged by cytotoxic treatments.

Results of experiments showed the effect of caspase 3 on tumor stroma. Data also indicate that there is significant influx of macrophages. Other effects include how the host immune system mobilizes against cancer, which is believed to be significantly influenced by how cells die (for example, necrosis vs apoptosis).

One of the important implications of findings by the present inventors is a novel and counter-intuitive approach to enhance cancer radiotherapy through caspase 3 inhibition. Surprisingly experimental results indicate a significant cancer treatment efficacy of adjuvant use of caspase inhibitors in radiotherapy. Another important discovery by the present inventors is the use of activated caspase 3 as a biomarker to predict tumor response to treatment, as supported by data from IHC analyses of human tumor samples.

To answer the question whether the mode of cell death affects the growth promoting signaling of caspase 3 from the dying cells, markers of apoptosis (activated caspase 3, TUNEL staining for DNA fragmentation), autophagy (increased accumulation of LC3-II isoform, which is essential for the formation of autophagosome), and necrosis (secreted HMGB1, a nuclear protein released into the extracellular space during necrosis) were evaluated after cellular exposure to 10 Gy of x-rays. Results indicated that in any given cell type, one can see at least two forms of cell death. In murine 4T1 cells, there were indicators of apoptosis (caspase 3 activation, PARP cleavage, and TUNEL staining), which were accompanied by the necrosis marker HMGB1 in the supernatants of cells, indicating co-existence of both types of cell death. There were also small but clear changes in the levels of autophagy marker LC3-II after radiation, accompanied by increases in perinuclear autophagosome staining In wild type MEF cells, apoptosis activation (as indicated by caspase 3 activation, PARP cleavage, and TUNEL staining) was accompanied by increasing amount of necrosis (secreted HMGB1) and a small but clear increase in LC3-II (autophagy marker) expression indicating co-existence of all three forms of cell death. In contrast, in caspase 3 deficient (Casp3^{-/-1}) MEF cells, there was a total absence of apoptosis markers (as indicated by caspase activation, PARP cleavage, and TUNEL staining) On the other hand, there was a significant increase in LC3-II accumulation, indicating increased autophagy in these cells. Interestingly, there was a clear decrease in expression of the necrosis marker HMGB1 secretion in these cells after irradiation. In human breast cancer cell line MCF-7, which has no expression of caspase 3, exposure to radiation induced no signs of apoptosis (PARP cleavage and TUNEL staining) However, there was an increase in autophagy (as indicated by increase in LC3-II expression) as well as necrotic (as indicated by strong induction of HMGB 1 secretion) cell death. In contrast, in the MCF-7CASP3 cell line, which has an exogenously expressed caspase 3 gene, radiation caused a significant increase in apoptosis (caspase 3 activation, PARP cleavage, and TUNEL staining) Induction of autophagy (as measured by LC3-II expression) appeared to be unchanged. However, induction of necrosis (as measured by HMGB1 in the supernatant) appeared to be weakened. Therefore, the status of apoptotic caspase 3 affected necrotic and autophagy pathways differentially in MEF cells and MCF-7 cells.

Results indicate that the status of caspase 3 expression can significantly affect the ways cells die. They also suggest the cells die through more than one way after radiation exposure. Furthermore, the absence of caspase 3 causes cells to shift their mode of death from apoptosis to necrosis with changes in autophagy status. In addition, different cells may shift their death modes in very different manners. Significantly, results show that irrespective of modes of cell death, caspase 3 is important for activating the growth-promoting signals emanating from the dying cells.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for predicting tumor recurrence in a subject comprising determining the amount of cells with caspase 3 in a cell sample of the subject, wherein at least 5% of the cell sample with caspase 3 is an indication of a likelihood of cancer recurrence.

2. The method of claim 1, wherein the sample cells comprises a tumor biopsy sample cells of the subject.

3. The method of claim 1, wherein the tumor is a cancer.

4. The method of claim 3, wherein the cancer comprises squamous cell carcinoma.

5. The method of claim 3, wherein the cancer comprises breast cancer, prostate cancer, colon cancer, melanoma, liver cancer, leukemia, lymphoma, and other solid tumors and blood-related cancer, or a combination thereof.

6. A method for determining a likelihood of recurrence of cancer in a patient, said method comprising determining the amount of cells having caspase 3 in a cell sample obtained from the patient's cancerous tissue or organ, wherein the amount of cells having caspase 3 in the cell sample is at least 5% is an indication of a likelihood of recurrence of cancer in the patient.

7. The method of claim 6, wherein the likelihood of recurrence of cancer in the patient having at least 5% of cells having caspase 3 in the cell sample is at least 70% compared to a control group with the same type of cancer and having less than 5% of cells having caspase 3.

8. The method of claim 6, wherein cancer comprises squamous cell carcinoma.

9. The method of claim 6, wherein cancer comprises breast cancer, prostate cancer, colon cancer, melanoma, liver cancer, leukemia, lymphoma, and other solid tumors and blood-related cancer, or a combination thereof.

10. A method for reducing the likelihood of cancer recurrence in a patient undergoing a cancer treatment, said method comprising administering to said patient a therapeutically effective amount of caspase 3 inhibitor.

11. The method of claim 10, wherein the caspase 3 inhibitor comprises Ac-DEVD-CHO; 5-[(S)-(+)-2-(Methoxymethyl)pyrrolidino]sulfonylisatin; Ac-DNLD-CHO; Z-D(OMe)E(Ome)VD(OMe)-FMK; Z-D(OMe)E(Ome)VD(OMe)-FMK; Ac-DMQD-CHO; Z-D(OMe)QMD (OMe)-FMK; 2-(4-Methyl-8-(morpholin-4-ylsulfonyl)-1,3-dioxo-1,3 -dihydro-2H-pyrrolo[3,4-c]quinolin-2-yl)ethyl; acetate Z-VAD-FMK; Ac-VAD-CHO; Boc-D-FMK, or a combination thereof.

12. The method of claim 10, wherein the caspase 3 inhibitor is administered to said patient during cancer treatment.

13. A method for treating cancer in a patient, said method comprising:
determining the amount of cells having caspase 3 activity from a cell sample obtained from the patient's cancerous tissue or organ prior to administering a cancer treatment;
administering a cancer treatment to the patient; and
administering a therapeutically effective amount of a caspase 3 inhibitor to the patient if at least 5% of cells in the cell sample have caspase 3 activity.

14. The method of claim 13, wherein the caspase 3 inhibitor is administered prior to administering the cancer treatment to the patient.

15. The method of claim 13, wherein the caspase 3 inhibitor is administered after administering the cancer treatment to the patient.

16. The method of claim 13, wherein the cancer treatment comprises chemotherapy, radiation therapy, or a combination thereof.
